(19) United States
(12) Patent Application Publication
(10) Pub. No.: US 2025/0331751 A1
(43) Pub. Date: Oct. 30, 2025

(54) EEG ANALYSIS METHOD AND APPARATUS FOR DIAGNOSING DEPRESSION

(71) Applicant: Chang-dong LEE, Wonju-si, Gangwon-do (KR)

(72) Inventor: Chang-dong LEE, Wonju-si, Gangwon-do (KR)

(21) Appl. No.: 18/551,183

(22) PCT Filed: Nov. 9, 2022
(86) PCT No.: PCT/KR2022/017497

§ 371 Date: Dec. 9, 2024

(30) Foreign Application Priority Data
Aug. 16, 2022 (KR) 10-2022-0102252

(51) Int. Cl. A61B 5/16; A61B 5/00; A61B 5/291; A61B 5/374; A61B 5/38

(52) U.S. Cl. CPC A61B 5/165; A61B 5/291; A61B 5/374; A61B 5/38; A61B 5/7275

(57) ABSTRACT

The present invention relates to an EEG analysis method and apparatus for diagnosing depression, and the method includes the steps of: generating sounds so that repetitive acoustic stimuli may be transferred to a subject; acquiring EEG signals measured from the subject in synchronization with the acoustic stimuli; calculating P300 latency using the acquired EEG signals; and providing depression diagnosis information for the subject on the basis of the calculated P300 latency.

### TECHNICAL FIELD

The present invention relates to a method of analyzing EEG as information for diagnosing depression or the like.

### BACKGROUND ART

The incidence of depression continues to increase in the advanced society as the life of modern people is getting more complex and diversified, and depression usually shows symptoms such as feeling of depression, loss of interest, loss of weight, disturbance of sleep, feeling of guilt, and the like, and in some cases, chronic fatigue or back pain may appear as a symptom.

Although depression tends to be easily regarded as a temporary emotional problem or personality rather than a mental disease, the depression is a serious mental disease that greatly affects the lives of a patient himself/herself and even family members around the patient, and results in suicide or the like in severe cases.

On the other hand, in the case of depression, 70 to 90% can be completely recovered when treated at a right time. However, as the depression is not recognized as a disease in most cases, patients do not visit hospitals and miss the chance of treatment.

Although diagnosis of depression has been performed mainly using the Hamilton Depression Rating Scale (HAM-D), the Beck Depression Scale (BDI), or the like as a clinical method, it is difficult to timely diagnose and appropriately treat the depression due to the negative perception or the like of psychiatric treatment.

Although results of researches showing that depression can be diagnosed using the characteristics of EEG signals are published recently with the advancement in brain science, measuring EEG in an unfamiliar environment such as a psychiatric clinic or laboratory has a problem of amplifying patient's anxiety.

Accordingly, instead of expensive EEG measuring equipment that requires specialized knowledge to diagnose depression, it needs to develop an EEG analysis apparatus for diagnosing depression, which has a simple structure and usability enough for individuals to purchase and use.

### DISCLOSURE OF INVENTION

### Technical Problem

Therefore, the present invention has been made in view of the above problems, and it is an object of the present invention to provide an EEG analysis method and apparatus for diagnosing depression.

### Technical Solution

To accomplish the above object, according to one aspect of the present invention, there is provided an EEG analysis method for diagnosing depression, the method comprising the steps of: generating sounds so that repetitive acoustic stimuli may be transferred to a subject; acquiring EEG signals measured from the subject in synchronization with the acoustic stimuli; calculating P300 latency using the acquired EEG signals; and providing depression diagnosis information for the subject on the basis of the calculated P300 latency.

According to another aspect of the present invention, there is provided an EEG analysis apparatus for diagnosing depression, the apparatus comprising: a sound generation unit for transferring repetitive acoustic stimuli to a subject; an electrode unit including a plurality of electrodes for measuring EEG signals of the subject; an EEG receiving unit for receiving the EEG signals measured from the subject through the electrode unit in synchronization with the acoustic stimuli; and an EEG analysis unit for calculating P300 latency using the received EEG signals, and configuring depression diagnosis information for the subject on the basis of the calculated P300 latency.

Meanwhile, at least some steps of the EEG analysis method for diagnosing depression may be implemented as a computer-readable recording medium that records a program to be executed on a computer, or may be provided as a program itself.

### Advantageous Effects

According to an embodiment of the present invention, it is possible to provide an EEG analysis apparatus for diagnosing depression, which has a simple structure and usability, and diagnoses depression in a way of calculating P300 latency by analyzing EEG signal responses measured in synchronization with repetitive acoustic stimuli, and providing depression diagnosis information for a subject.

In addition, information may be provided by combining results of ATR (Alpha-Theta Ratio) analysis and alpha asymmetry analysis, together with P300 latency, so that possibility of depression and other psychiatric disorders may be diagnosed.

### BRIEF DESCRIPTION OF THE DRAWINGS

FIG. 1 is a block diagram showing an embodiment of the overall configuration of an EEG analysis apparatus for diagnosing depression according to the present invention.

FIG. 2 is a view showing an embodiment of the arrangement of a plurality of electrodes included in an electrode unit.

FIG. 3 is a flowchart illustrating an embodiment of an EEG analysis method for diagnosing depression according to the present invention.

FIG. 4 is a view showing an embodiment of an acoustic stimulus transferred to a subject.

FIG. 5 is a view for explaining an embodiment of a method of recording EEG signals for calculation of P300 latency.

FIG. 6 is a view for explaining an embodiment of a method of calculating P300 latency using repeatedly recorded EEG signals.

FIG. 7 is a flowchart illustrating another embodiment of an EEG analysis method for diagnosing depression according to the present invention.

FIG. 8 is a flowchart illustrating an embodiment of an ATR analysis method.

FIG. 9 is a flowchart illustrating an embodiment of an alpha asymmetry analysis method.

### BEST MODE FOR CARRYING OUT THE INVENTION

Hereinafter, an EEG analysis method and apparatus for diagnosing depression according to an embodiment of the present invention will be described in detail with reference to the accompanying drawings.

In describing the present invention below, when it is determined that a detailed description of a related known function or configuration may unnecessarily obscure subject matters of the present invention, the detailed description will be omitted. In addition, terms described below are terms defined in consideration of the functions in the present invention, which may vary according to the intention of a user or an operator, customs, or the like. Therefore, definitions thereof should be made based on the contents disclosed throughout the present invention.

In addition, in order to efficiently describe the technical components constituting the present invention, in the preferred embodiments of the present invention embodied hereinafter, functional configurations of the system already provided in each system or commonly provided in the technical field of the present invention are omitted as much as possible, and it will be described focusing on the functional configurations that should be additionally provided for the present invention.

Those skilled in the art may easily understand the functions of conventionally used components among the functional configurations not shown and omitted below, and may also clearly understand the relationship between the components omitted as described above and components added for the present invention.

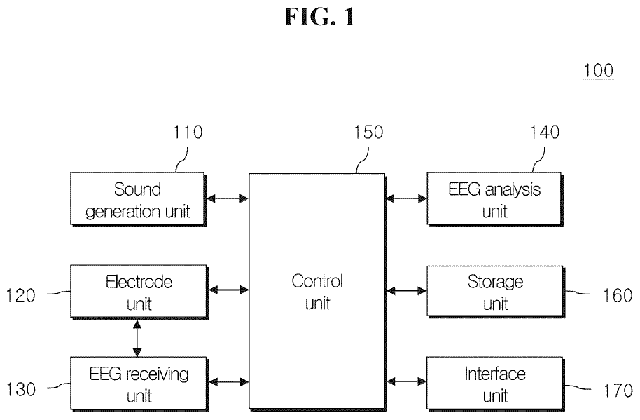

FIG. 1 is a block diagram showing an embodiment of the overall configuration of an EEG analysis apparatus for diagnosing depression according to the present invention, and the illustrated EEG analysis apparatus 100 may be configured to include a sound generation unit 110, an electrode unit 120, an EEG receiving unit 130, an EEG analysis unit 140, a control unit 150, a storage unit 160, and an interface unit 170.

Referring to FIG. 1, the sound generation unit 110 is for transferring repetitive acoustic stimuli to a subject, and may be configured to include an acoustic stimulus generator for generating acoustic stimuli, and a headphone for outputting the acoustic stimuli.

The electrode unit 120 includes a plurality of electrodes for measuring EEG signals of a subject, and the plurality of electrodes may be classified into a ground electrode, a reference electrode, and an active electrode.

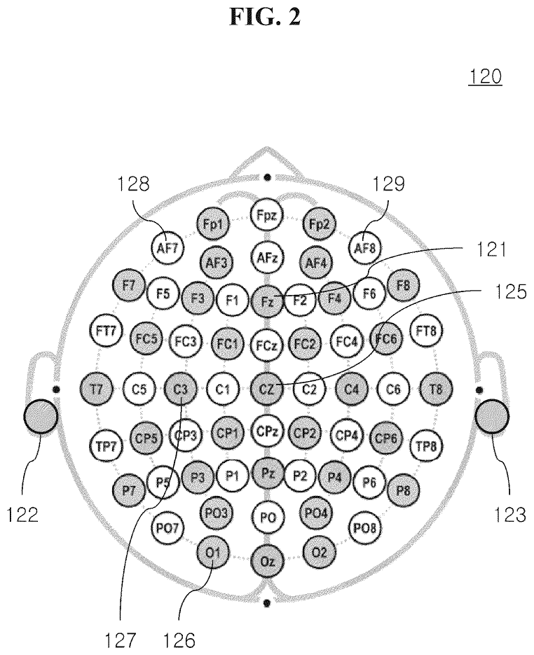

According to an embodiment of the present invention, as shown in FIG. 2, the electrode unit 120 may include a ground electrode 121 disposed in the median frontal region Fz of a subject, and reference electrodes 122 and 123 disposed on the left and right earlobes.

In addition, the electrode unit 120 may include a plurality of active electrodes 125 to 129 for measuring EEG signals that will be used for diagnosis of depression.

For example, a first electrode 125 may be disposed in the median center Cz, a second electrode 126 may be disposed in the left occipital lobe O1, a third electrode 127 may be disposed in the left central column C3, a fourth electrode 128 may be disposed in the left frontal lobe AF7, and a fifth electrode 129 may be disposed in the right frontal lobe AF8.

Although the electrode unit 120 may be configured to include a total of eight electrodes including the ground electrode 121, the two reference electrodes 122 and 123, and the five active electrodes 125 to 129 as described above, the present invention is not limited thereto, and some electrodes may be omitted or one or more electrodes disposed in other regions may be added as needed.

The EEG receiving unit 130 receives EEG signals measured from a subject through the electrode unit 120 while acoustic stimuli are transferred to the subject through the sound generation unit 110.

The EEG analysis unit 140 may calculate P300 latency using the received EEG signals, and configure depression diagnosis information for the subject on the basis of the calculated P300 latency.

Here, the depression diagnosis information may include various information about the results of analyzing the EEG signals, for example, whether the subject has depression (or is highly likely have depression), a degree of depression of the subject, information needed for an expert such as a doctor or the like to diagnose depression, and the like.

For example, when the calculated P300 latency exceeds a preset reference value (e.g., 320 ms), the EEG analysis unit 140 may determine that the depression diagnosis information may indicate that the subject is highly likely to have depression (or may correspond to depression).

On the other hand, the P300 latency is previously divided into a plurality of sections (e.g., Mild/Moderate/Severe/Very severe) representing different degrees of depression, and in this case, the depression diagnosis information may include information on a section to which the calculated P300 latency belongs among the plurality of sections.

According to another embodiment of the present invention, the EEG analysis unit 140 may calculate an ATR (Alpha-Theta Ratio) obtained by dividing the power of alpha wave in the EEG signals by the power of theta wave, convert the calculated ATR (Alpha-Theta Ratio) into a probability score, and include the converted probability score in depression diagnosis information as a result of the depression diagnosis.

According to still another embodiment of the present invention, the EEG analysis unit 140 may calculate asymmetry by comparing the power of at least one among the alpha wave and the beta wave, make an analysis on the basis of the calculated alpha/theta asymmetry, and include the analyzed depression diagnosis result in the depression diagnosis information.

Meanwhile, according to still another embodiment of the present invention, the EEG analysis unit 140 may configure depression diagnosis information of the subject by performing analysis comprehensively considering the P300 latency, the ATR (Alpha-Theta Ratio), and the alpha/beta asymmetry calculated from the EEG signals as described above.

The control unit 150 may control the overall operation of the depression diagnosis apparatus 100 as described above, for example, the operation of each component, and interworking between the components.

The storage unit 160 may store measured EEG signal data, data calculated as a signal analysis result (e.g., P300 latency, ATR (Alpha-Theta Ratio), alpha/beta asymmetry, etc.), depression diagnosis information, and the like.

In addition, the interface unit 170 may include a user input unit having buttons or the like for receiving input from a user, a display unit or a speaker for transferring information to the user, and a communication unit for providing data, such as depression diagnosis information or the like, to the outside.

Although the EEG analysis apparatus 100 according to an embodiment of the present invention as described above may have a form mounted on the head of a subject, the present invention is not limited thereto, and for example, components other than the electrode unit 120 for measuring the EEG of the subject may be configured as one or two or more separate modules.

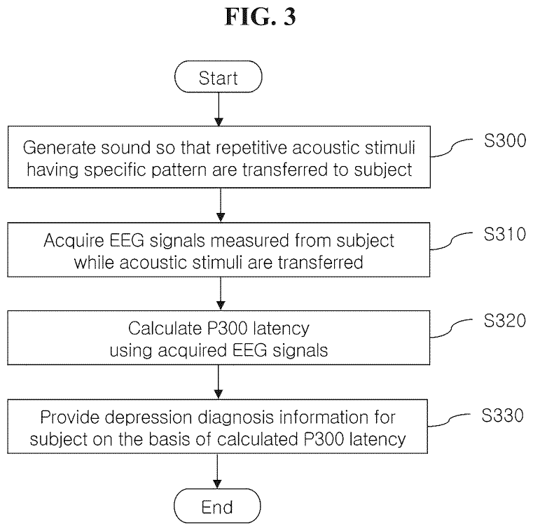

FIG. 3 is a flowchart illustrating an embodiment of an EEG analysis method for diagnosing depression according to the present invention, and in the EEG analysis method shown in FIG. 3, descriptions the same as those described with reference to FIGS. 1 and 2 will be omitted hereinafter.

Referring to FIG. 3, the EEG analysis apparatus 100 generates sounds so that repetitive acoustic stimuli may be transferred to the subject (step S300).

For example, acoustic stimuli used for auditory evoked potential (AEP) may be transferred to the subject at step S300.

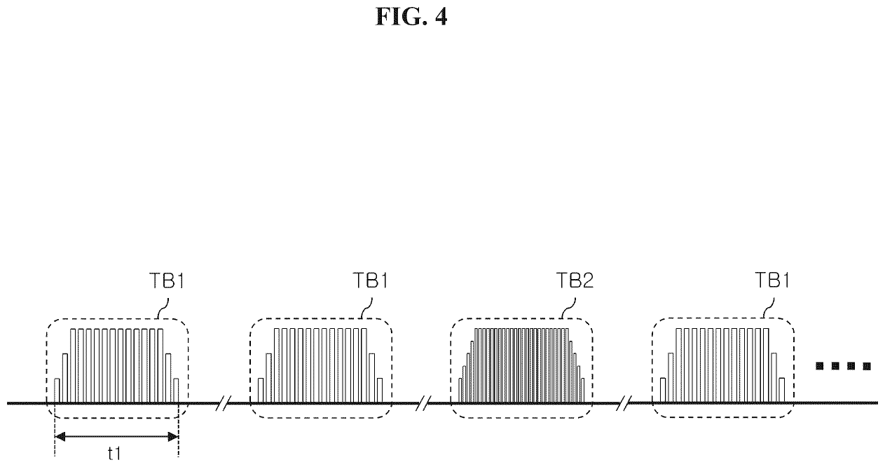

Referring to FIG. 4, a first tone burst TB1 and a second tone burst TB2 may be sequentially generated and transferred to the subject through a headphone, and although the stimulus duration t1 of the first tone burst TB1 and the second tone burst TB2 may be 200 ms, the present invention is not limited thereto.

Here, the frequency and occurrence interval of the first tone burst TB1 may be different those of the second tone burst TB2.

For example, the frequency of the first tone burst TB1 may be lower than the frequency of the second tone burst TB2, and the frequencies may be 500 Hz and 1,000 Hz, respectively.

Meanwhile, the occurrence interval of the first tone burst TB1 may be larger than the occurrence interval of the second tone burst TB2, and accordingly, the first tone burst TB1 is referred to as a frequent tone burst, and the second tone burst TB2 may be referred to as a rare tone burst.

The EEG analysis process according to an embodiment of the present invention may be carried out in a quiet and slightly dark room while the subject is sitting in a comfortable chair in an awake state, and performed as the subject counts the number of occurrences of the second tone burst TB2 of a high frequency while a total of 300 acoustic stimuli, including the first tone burst TB1 and the second tone burst TB2, are transferred.

To this end, the second tone burst TB2 is randomly generated so that the subject may not predict, and therefore, the frequency and interval of generating the second tone burst TB2 may be changed in each examination.

The EEG analysis apparatus 100 acquires EEG signals measured from the subject while the acoustic stimuli are transferred (step S310).

At step S310, the EEG analysis apparatus 100 may measure the EEG signals of the subject using at least some of the plurality of electrodes disposed as shown in FIG. 2.

For example, the EEG analysis apparatus 100 may repeatedly record and store the EEG signals measured through the first electrode 125 in synchronization with the generation time point of the repetitive acoustic stimuli transferred to the subject at step S300.

That is, the EEG analysis apparatus 100 may record the EEG signals measured through the active electrode 125 disposed in the median center Cz using the electrode 121 disposed in the median frontal region Fz as a ground electrode and the electrodes 122 and 123 disposed on both earlobes as reference electrodes.

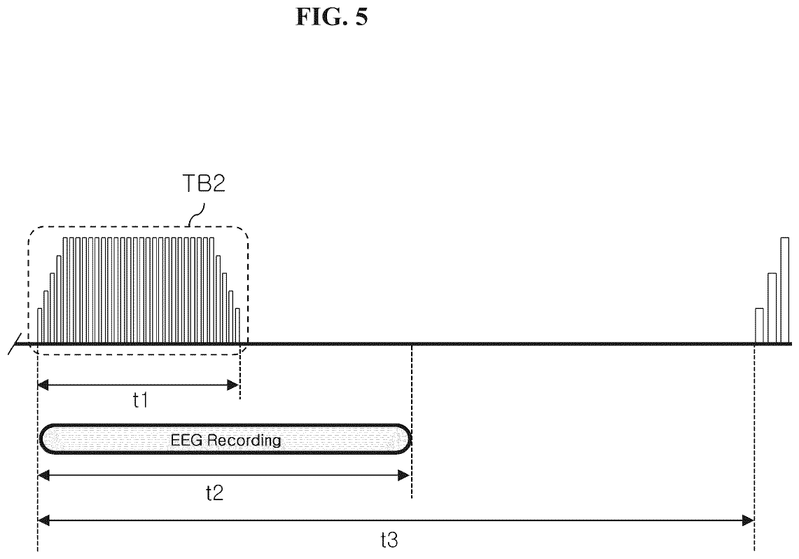

Referring to FIG. 5, a randomly generated second tone burst TB2 has a frequency of 1,000 Hz and may be output during a stimulus duration t1 of 200 ms.

Meanwhile, in synchronization with the generation time point of the second tone burst TB2, the EEG signals measured through the first electrode 125 may be recorded and stored during a recording time t2 of 512 ms, from the time point of starting generation of the second tone burst TB2.

In addition, a next acoustic stimulus, e.g., the first tone burst TB1, may be generated again for 200 ms with a stimulus generation interval t3 of 1.1 s from the time point of starting generation of the second tone burst TB2.

Whenever the second tone burst TB2 is generated, the EEG signals measured through the first electrode 125 may be repeatedly recorded and stored in the storage unit 160 for 512 ms from the time point of starting generation of the second tone burst TB2 as described above.

Thereafter, the EEG analysis apparatus 100 calculates P300 latency using the EEG signals acquired at step S310 (step S320).

For example, the EEG analysis apparatus 100 may calculate an average after removing artifacts of the EEG signals repeatedly recorded and stored in synchronization with the acoustic stimulus, and calculate P300 latency using the calculated average value.

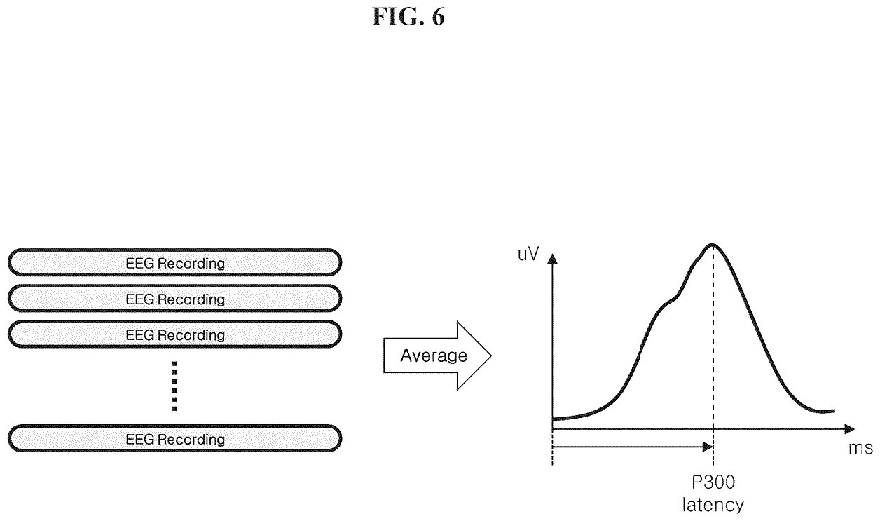

Referring to FIG. 6, the P300 latency may be obtained by averaging the EEG signals repeatedly recorded whenever the second tone burst TB2 is generated as described above, and measuring the time until the power of the averaged EEG signal reaches a peak.

The EEG analysis apparatus 100 provides depression diagnosis information for the subject on the basis of the P300 latency calculated at step S320 (step S330).

For example, at step S330, the EEG analysis apparatus 100 may determine that the subject corresponds to depression when the calculated P300 latency exceeds a preset reference value of 320 ms.

Alternatively, the degree of depression is classified into 4 levels of Mild, Moderate, Severe, and Very severe, and a range of P300 latency corresponding to each level may be set in advance.

In this case, the EEG analysis apparatus 100 may confirm a section to which the P300 latency calculated at step S320 belongs among the 4 levels, and then provide a corresponding depression level as depression diagnosis information.

Meanwhile, at step S330, the EEG analysis apparatus 100 may provide the depression diagnosis information analyzed based on the P300 latency to the subject through a display provided in the apparatus, or may transfer the depression diagnosis information to an external device using wired/wireless communication.

According to still another embodiment of the present invention, the EEG analysis apparatus 100 may configure depression diagnosis information for the subject by combining and analyzing various data that can be calculated from the EEG signals of the subject, in addition to the P300 latency.

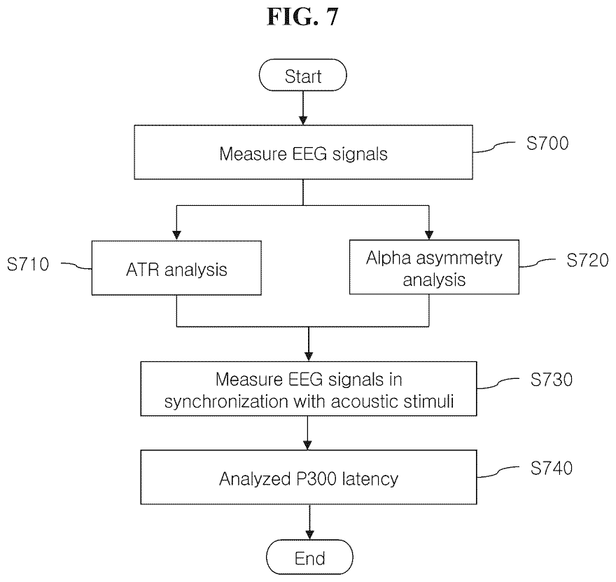

FIG. 7 is a flowchart illustrating another embodiment of an EEG analysis method for diagnosing depression according to the present invention, and in the EEG analysis method shown in FIG. 7, descriptions the same as those described with reference to FIGS. 1 to 6 will be omitted below.

Referring to FIG. 7, EEG signals from the subject are measured and recorded (step S700), and ATR analysis (step S710) and alpha asymmetry analysis (step S720) are performed using the measured EEG signals.

The process of measuring EEG signals at step S700 may be carried out in a quiet and slightly dark room while the subject is sitting in a comfortable chair in an awake state.

Meanwhile, the ground electrode 121 and the two reference electrodes 122 and 123 disposed as shown in FIG. 2 are used for the ATR analysis at step S710, and EEG signals measured through the second electrode 126 disposed on the left occipital lobe O1 and the third electrode 127 disposed in the left central column C3 may be recorded.

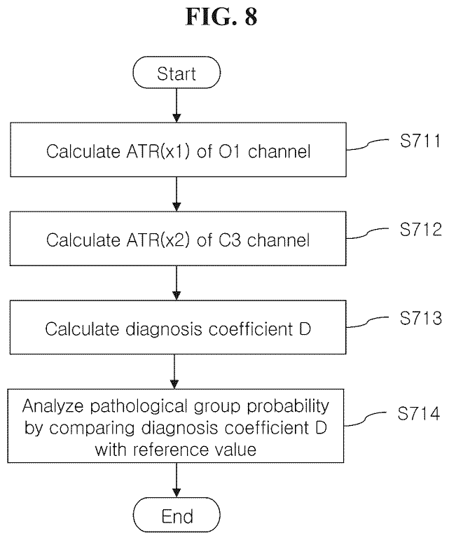

In the ATR analysis, as shown in FIG. 8, ATR(x1) of the left occipital lobe O1 channel and the ATR(x2) of the left central column C3 channel are calculated (steps S711 and S712).

For example, at step S711, power of alpha wave and power of theta wave are acquired from the EEG signals measured through the second electrode 126 disposed in the left occipital lobe O1, and ATR(x1) may be calculated by dividing the acquired power of alpha wave by the power of theta wave.

Meanwhile, at step S712, power of alpha wave and power of theta wave are acquired from the EEG signals measured through the third electrode 127 disposed in the left central column C3, and ATR(x2) may be calculated by dividing the acquired power of alpha wave by the power of theta wave.

Next, a diagnosis coefficient D is calculated using the ATR values calculated at steps S711 and S712.

For example, the diagnosis coefficient D may be calculated by applying Equation 1 shown below to the ATR(x1) of the left occipital lobe O1 channel and the ATR(x2) of the left central column C3 channel.

Diagnosis coefficient (D)=12*(x1)+18*(x2) [Equation 1]

Thereafter, the pathological group probability is analyzed by comparing the diagnosis coefficient D calculated at step S713 with a preset reference value (step S714).

For example, when the calculated diagnosis coefficient D is lower than 33, it may be pathologically analyzed that the probability of belonging to a cognitive disorder group such as dementia, Alzheimer's disease, or the like is high, and when the diagnosis coefficient D is 33 or higher, it may be pathologically analyzed that the probability of belonging to the cognitive disorder group is low.

In addition, the ground electrode 121 and the two reference electrodes 122 and 123 disposed as shown in FIG. 2 are used for the alpha asymmetry analysis of step S720, and EEG signals measured through the fourth electrode 128 disposed in the left frontal lobe AF7 and the fifth electrode 129 disposed in the right frontal lobe AF8 may be recorded.

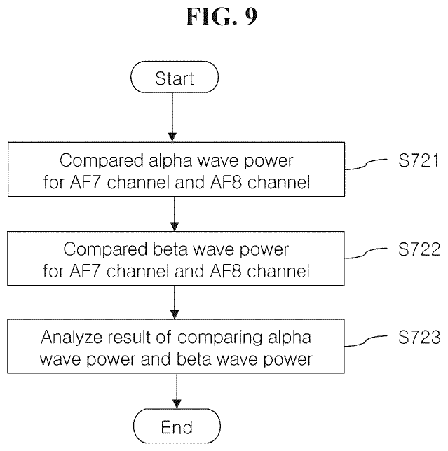

In the alpha asymmetry analysis, the power of alpha wave is compared for the left frontal lobe AF7 channel and the right frontal lobe AF8 channel as shown in FIG. 9 (step S721).

For example, left frontal lobe alpha wave power AF7 Alpha is acquired from the EEG signal measured through the fourth electrode 128 disposed in the left frontal lobe AF7, and right frontal lobe alpha wave power AF8 Alpha is acquired from the EEG signal measured through the fifth electrode 129 disposed in the right frontal lobe AF8, and it can be confirmed whether the left frontal lobe alpha wave power AF7 Alpha is greater than the right frontal lobe alpha wave power AF8 Alpha.

In addition, the power of beta wave is compared for the left frontal lobe AF7 channel and the right frontal lobe AF8 channel (step S722).

For example, left frontal lobe beta wave power AF7 Beta is acquired from the EEG signal measured through the fourth electrode 128 disposed in the left frontal lobe AF7, and right frontal lobe beta wave power AF8 Beta is acquired from the EEG signal measured through the fifth electrode 129 disposed in the right frontal lobe AF8, and it can be confirmed whether the right frontal lobe beta wave power AF8 Beta is greater than the left frontal lobe beta wave power AF7 Beta.

Thereafter, depression-related information is analyzed using the result of alpha wave power comparison at step S721 and the result of beta wave power comparison at step S722 (step S723).

For example, when the left frontal lobe alpha wave power AF7 Alpha is greater than the right frontal lobe alpha wave power AF8 Alpha, whether the right frontal lobe beta wave power AF8 Beta is greater than the left frontal lobe beta wave power AF7 Beta is confirmed, and when the right frontal lobe beta wave power AF8 Beta is greater than the left frontal lobe beta wave power AF7 Beta, it may be analyzed that the probability of depression accompanied by nervousness is high, when the right frontal lobe beta wave power AF8 Beta is not greater than the left frontal lobe beta wave power AF7 Beta, it may be analyzed that the probability of depression accompanied by despair is high.

On the other hand, when the left frontal lobe alpha wave power AF7 Alpha is not greater than the right frontal lobe alpha wave power AF8 Alpha, it may be analyzed that the probability of depression is low.

As described above, after the ATR analysis (step S710) and the alpha asymmetry analysis (step S720) are performed, analysis using P300 latency may be performed.

To this end, EEG signals are measured in synchronization with acoustic stimuli (step S730), and P300 latency is calculated and analyzed using the measured EEG signals (step S740).

Although analysis is performed using the EEG signals measured through active electrodes at specific locations without a separate acoustic stimulus as described above in the case of the ATR analysis (step S710) and the alpha asymmetry analysis (step S720), acoustic stimuli are repeatedly transferred to the subject for the analysis of P300 latency (step S740), and P300 latency may be calculated using the EEG signals measured and recorded in synchronization with the acoustic stimuli.

Since the method of calculating P300 latency by repeatedly measuring and recording EEG signals in synchronization with acoustic stimuli may be the same as those described with reference to FIGS. 3 to 6, detailed description thereof will be omitted.

At step S740, when the P300 latency is calculated as being larger than 320 ms, which is the reference value, it can be analyzed that the probability of depression of the subject is high.

Although an embodiment of a method of analyzing EEG through ATR analysis (step S710), alpha asymmetry analysis (step S720), and P300 latency analysis (step S740) has been described above, according to another embodiment of the present invention, information for diagnosing depression can be provided by integrating the analysis results as described above.

Hereinafter, embodiments of a method of providing information for diagnosing depression by integrating analysis results will be described.

TABLE 1

| Analysis items | Results |
|---|---|
| ATR diagnosis coefficient (D) | 36 |
| Frontal lobe alpha wave power | Left > Right |
| Frontal lobe beta wave power | Left = Right |
| P300 latency | 300 ms |

In the case of the analysis result as shown in Table 1, the probability of depression is low as the P300 latency is lower than 320 ms, which is normal, but the frontal lobe alpha wave comparison result (power of the left frontal lobe alpha wave is greater than the power of the right frontal lobe alpha wave) indicates depression, and since the ATR diagnosis coefficient D is 33 or higher, which is normal, it may indicate that the subject does not have depression, but is in a depressed state.

TABLE 2

| Analysis items | Results |
|---|---|
| ATR diagnosis coefficient (D) | 34 |
| Frontal lobe alpha wave power | Left > Right |
| Frontal lobe beta wave power | Right > Left |
| P300 latency | 310 ms |

In the case of the analysis result as shown in Table 2, the P300 latency is lower than 320 ms, which is normal, but somewhat slow, and the frontal lobe alpha wave comparison result (power of the left frontal lobe alpha wave is greater than the power of the right frontal lobe alpha wave) and the frontal lobe beta wave comparison result (power of the right frontal lobe alpha wave is greater than the power of the left frontal lobe alpha wave) indicate depression and tension, and since the ATR diagnosis coefficient D is 33 or higher, which is normal, it may indicate that the subject does not have depression, but shows a depressed state due to stress and tension and may be developed into depression.

TABLE 3

| Analysis items | Results |
|---|---|
| ATR diagnosis coefficient (D) | 30 |
| Frontal lobe alpha wave power | Left > Right |
| Frontal lobe beta wave power | Right = Left |
| P300 latency | 340 ms |

In the case of the analysis result as shown in Table 3, the P300 latency is 340 ms (exceeds 320 ms), showing depression, and the frontal lobe alpha wave comparison result (power of the left frontal lobe alpha wave is greater than the power of the right frontal lobe alpha wave) indicates depression, and since the ATR diagnosis coefficient D is 30 (lower than 33), showing that the cognitive ability is lowered significantly, it may indicate that the subject may have depression accompanied by dementia.

TABLE 4

| Analysis items | Results |
|---|---|
| ATR diagnosis coefficient (D) | 30 |
| Frontal lobe alpha wave power | Left = Right |
| Frontal lobe beta wave power | Right < Left |
| P300 latency | 330 ms |

In the case of the analysis result as shown in Table 4, the P300 latency is 330 ms (exceeds 320 ms), showing depression, but the result of comparing the frontal lobe alpha wave and beta wave is normal, and since the cognitive ability is lowered as the ATR diagnosis coefficient D is 30 (lower than 33), it may indicate that the subject may have dementia.

Although embodiments of the EEG analysis method for diagnosing depression have been described above, the present invention is not limited thereto, and various information acquired in the EEG analysis process may be provided through the EEG analysis apparatus according to an embodiment of the present invention, and experts such as doctors or the like may use the information provided through the EEG analysis apparatus to diagnose various psychiatric and neurological diseases such as depression or other cognitive disorders such as dementia.

The methods according to an embodiment of the present invention described above may be produced as a program executed on a computer. In addition, the program may be stored in a computer-readable recording medium, and examples of the computer-readable recording medium include ROM, RAM, CD-ROM, magnetic tape, floppy disk, and optical data storage devices.

The computer-readable recording medium is distributed in computer systems connected through a network, and computer-readable codes may be stored and executed in a distributed manner. In addition, function programs, codes, and code segments for implementing the method can be easily inferred by programmers in the art to which the present invention belongs.

In addition, although the preferred embodiments of the present invention have been shown and described above, the present invention is not limited to the specific embodiments described above, and various modifications can be made by those skilled in the art without departing from the subject matters of the present invention claimed in the claims, and these modified embodiments should not be individually understood from the technical spirit or perspective of the present invention.

1. An EEG analysis method for diagnosing depression, the method comprising the steps of:
generating sounds so that repetitive acoustic stimuli may be transferred to a subject;
acquiring EEG signals measured from the subject in synchronization with the acoustic stimuli;
calculating P300 latency using the acquired EEG signals; and
providing depression diagnosis information for the subject on the basis of the calculated P300 latency.

2. The method according to claim 1, wherein the EEG signals are acquired using a first electrode disposed in a median center region of the subject.

3. The method according to claim 1, wherein when the calculated P300 latency exceeds a reference value, the depression diagnosis information indicates that the subject is highly likely to have depression.

4. The method according to claim 1, wherein the P300 latency is divided into a plurality of sections representing different degrees of depression, and the depression diagnosis information includes information on a section to which the calculated P300 latency belongs among the plurality of sections.

5. The method according to claim 1, further comprising the steps of:
acquiring the EEG signals using a second electrode and a third electrode respectively disposed in a left occipital lobe region and a left central column region of the subject;
calculating a first ratio by dividing power of alpha wave by power of theta wave for the EEG signal acquired through the second electrode;
calculating a second ratio by dividing power of alpha wave by power of theta wave for the EEG signal acquired through the third electrode; and
calculating a diagnosis coefficient using the calculated first and second ratios.

6. The method according to claim 1, further comprising the steps of:
acquiring the EEG signals using a fourth electrode and a fifth electrode respectively disposed in a left frontal lobe region and a right frontal lobe region of the subject;
comparing alpha wave power of the EEG signal acquired through the fourth electrode and alpha wave power of the EEG signal acquired through the fifth electrode; and
comparing beta wave power of the EEG signal acquired through the fourth electrode and beta wave power of the EEG signal acquired through the fifth electrode.

7. An EEG analysis apparatus for diagnosing depression, the apparatus comprising:
a sound generation unit for transferring repetitive acoustic stimuli to a subject;
an electrode unit including a plurality of electrodes for measuring EEG signals of the subject;
an EEG receiving unit for receiving the EEG signals measured from the subject through the electrode unit in synchronization with the acoustic stimuli; and
an EEG analysis unit for calculating P300 latency using the received EEG signals, and configuring depression diagnosis information for the subject on the basis of the calculated P300 latency.

8. The apparatus according to claim 7, wherein the electrode unit includes:
a ground electrode disposed in a median frontal region of the subject;
reference electrodes respectively disposed in the left and right earlobe regions;
a first electrode disposed in a median center region;
a second electrode disposed in a left occipital lobe region;
a third electrode disposed in a left central column region;
a fourth electrode disposed in a left frontal lobe region; and
a fifth electrode disposed in the right frontal lobe region.

9. The apparatus according to claim 8, wherein the EEG analysis unit calculates a first ratio by dividing power of alpha wave by power of theta wave for the EEG signal acquired through the second electrode, calculates a second ratio by dividing power of alpha wave by power of theta wave for the EEG signal acquired through the third electrode, and calculates a diagnosis coefficient using the calculated first and second ratios.

10. The apparatus according to claim 8, wherein the EEG analysis unit compares alpha wave power of the EEG signal acquired through the fourth electrode and alpha wave power of the EEG signal acquired through the fifth electrode, and compares beta wave power of the EEG signal acquired through the fourth electrode and beta wave power of the EEG signal acquired through the fifth electrode.
